(19) United States
(12) Patent Application Publication
(10) Pub. No.: US 2024/0055123 A1
(43) Pub. Date: Feb. 15, 2024

(54) A METHOD FOR ESTIMATING NARROWINGS IN ARTERIES OF A HEART AND AN APPARATUS THEREOF

(71) Applicants: Ida Arvidsson, Höör (SE); Miguel Ochoa-Figueroa, Sturefors (SE)

(72) Inventors: Ida ARVIDSSON, Höör (SE); Miguel OCHOA-FIGUEROA, Sturefors (SE); Anders HEYDEN, Dalby (SE); Niels Christian OVERGAARD, Kävlinge (SE); Karl ÅSTRÖM, Lund (SE)

(21) Appl. No.: 18/259,048

(22) PCT Filed: Dec. 16, 2021
(86) PCT No.: PCT/SE2021/051269

§ 371 Date: Jun. 22, 2023

(30) Foreign Application Priority Data
Dec. 23, 2020 (SE) 2030374-9

(51) Int. Cl. G16H 50/20; A61B 6/00; G16H 30/40; G06T 7/00

(52) U.S. Cl. CPC G16H 50/20; A61B 6/504; G16H 30/40; G06T 7/00; G06T 2207/30104

(57) ABSTRACT

It is presented a computer-implemented method for estimating narrowings in arteries of a heart based on a sample myocardial perfusion imaging (S-MPI) data set using an artificial neural network (ANN). The method comprises receiving, in a data processing apparatus, the S-MPI data and a request, determining, in the data processing apparatus, an estimate quantitative coronary angiography (E-QCA) data set based on the S-MPI data set using the ANN, wherein the ANN is trained using a reference MPI (R-MPI) data set, a reference quantitative coronary angiography (R-QCA) data set and a reference invasive measurement (R-IM) data set, wherein each record in the R-MPI data set has a corresponding record in the R-QCA data set and a corresponding record in the R-IM data set, respectively, and transmitting, from the data processing apparatus, the E-QCA data set in response to the request.

### TECHNICAL FIELD

The invention generally relates to assessing image data depicting arteries of a heart to identify narrowings in the arteries. More particularly, it is related to a computer-implemented method for estimating such narrowings, a data processing apparatus and a computer program product.

### BACKGROUND

Myocardial perfusion imaging (MPI) is one of the most common cardiological examinations performed for diagnosis and risk assessment in patients with suspected coronary artery disease (CAD), providing valuable information on ischemia, myocardial injuries and left ventricular ejection fraction, among others. The technique has seen improvements in recent years with the introduction of cadmium-zinc-telluride (CZT) technology, allowing to perform the scan in shorter times and low dose radiotracer protocols, among other advantages, achieving high diagnostic performance at the same time.

Patients are usually referred to this study by the cardiologist, who commonly performs a clinical evaluation for ischemia, usually based on the guidelines for the diagnosis and management of chronic coronary syndromes from the European Society of Cardiology (ESC). This information is of great value to nuclear medicine physicians reading the MPI images in order to have a clinical scenario of the patient.

Patients who are thought to have CAD in the MPI study will be further examined, and eventually treated, by means of invasive coronary angiography (ICA). In order to avoid observatory dependent errors and achieve reproducibility, the degree of coronary artery stenosis can be evaluated by means of quantitative coronary angiography (QCA).

Since the pioneer work of Fujita et al in the 1990's, see Fujita H, Katafuchi T, Uehara T, et al. Application of artificial neural network to computer-aided diagnosis of coronary artery disease in myocardinal SPECT bull's-eye images. J Nuc/Med 1992, 33(2), 272-276, artificial neural networks (ANNs) have been used to evaluate MPI images. ANNs and MPI technology have evolved and recent studies in the field show promising results.

Even though that the development of assessing image data depicting arteries have progressed during the last decades, there are still improvements to be made. In particular, how to train and use artificial neural networks to reliably identify severe narrowings in heart arteries is highly relevant since such a software tool, capable of assessing the image data, may result in both improved time efficiency, i.e. that less time will be needed for examining a patient, and also in improved reliability, i.e. less risk of incorrect assessment of the image data.

### SUMMARY

It is an object of the invention to at least partly overcome one or more of the above-identified limitations of the prior art. In particular, it is an object to improve how image data, such as MPI data, is used for detecting narrowings in arteries in a heart. More particularly, it is an object to find an improved way of using ANNs for this purpose. The improvement may in this context may be detecting the narrowings faster, i.e. reducing time needed per patient, which will provide the possibility to shorten waiting times for patients, but also to provide information to the physician quickly in an emergency situation. In addition or alternatively, the improvement may also be that a more reliable assessment can be made, i.e. that the risk of heart failure can be estimated with a higher certainty, but also in that a position of the narrowing(s) can be estimated.

According to a first aspect it is provided a computer-implemented method for estimating narrowings in arteries of a heart based on a sample myocardial perfusion imaging (S-MPI) data set using an artificial neural network (ANN), said method comprising receiving, in a data processing apparatus, the S-MPI data set and a request, determining, in the data processing apparatus, an estimate quantitative coronary angiography (E-QCA) data set based on the S-MPI data set using the ANN, wherein the ANN is trained using a reference MPI (R-MPI) data set, a reference quantitative coronary angiography (R-QCA) data set and a reference invasive measurement (R-IM) data set, wherein each record in the R-MPI data set has a corresponding record in the R-QCA data set and a corresponding record in the R-IM data set, respectively, and transmitting, from the data processing apparatus, the E-QCA data set in response to the request.

By training the ANN with a combination of the R-MPI data set, the R-QCA data set and the R-IM data set, the ANN can reliably and fast provide the E-QCA data set in response to the received S-MPI data set.

The R-IM data set may comprise a reference fractional flow reserve (R-FFR) data set, a reference invasive coronary angiography R-ICA data set and/or an reference instant wave-free ratio (R-iFR) data set.

The ANN may be trained in two steps involving a first training step in which the R-MPI data set is combined with the R-ICA data set such that an intermediately trained ANN is generated, and a second training step in which the R-iFR data set and/or R-FFR data set is combined with the intermediately trained ANN such that the ANN is generated.

An advantage with this two-step approach is that the training of the ANN can be made such that reliable assessments can be made based on the S-MPI data set.

The ANN may be a convolutional neural network (CNN).

A patient-specific auxiliary parameter data set may be comprised in an additional input layer, said method further comprising concatenating a convolutional part of the ANN with the additional input layer, wherein the convolutional part is used for extracting features from the S-MPI data set.

An advantage with using the patient specific auxiliary data set in this way is that an improved feature extraction can be achieved, which in turn provides for a better assessment of the S-MPI data set.

The patient-specific auxiliary parameter data set may comprise age, sex, angina pectoris, and dyspnea. In addition or instead, the patient-specific auxiliary parameter data set may comprise smoking habits, systolic blood pressure, total cholesterol and place of residence.

By having the patient specific auxiliary parameter data set based on this information, the feature extraction can be adapted such that a more reliable end result can be achieved.

The ANN may comprise at least 7 layers.

By using seven layers or more the ANN can be trained such that reliable assessments of the S-MPI data set can be achieved.

The S-MPI data set may be captured by using a cadmium zinc telluride (CZT) camera, a gamma camera, and/or a positron emission tomography (PET) camera.

The E-QCA data set may comprise a left anterior artery (LAD) QCA data sub-set, a circumflex artery (LCx) QCA data sub-set and a right coronary artery (RCA) QCA data sub-set.

The S-MPI data set and the R-MPI data set may comprise stress MPI data.

According to a second aspect it is provided a data processing apparatus for estimating narrowings in arteries of a heart based on a sample myocardial perfusion imaging (S-MPI) data set using an artificial neural network (ANN), said apparatus comprising a data receiver configured to receive the S-MPI data set and a request, a processor configured to determine an estimate quantitative coronary angiography (E-QCA) data set based on the S-MPI data set using the ANN, wherein the ANN is trained using a reference MPI (R-MPI) data set, a reference quantitative coronary angiography (R-QCA) data set and a reference invasive measurement (R-IM) data set, wherein each record in the R-MPI data set has a corresponding record in the R-QCA data set and a corresponding record in the R-IM data set, respectively, and a data transmitter configured to transmit the E-QCA data set in response to the request.

For this second aspect, the advantages and features presented above with respect to the first aspect also apply.

The R-IM data set may comprise a reference fractional flow reserve (R-FFR) data set, a reference invasive coronary angiography (R-ICA) data set and/or an reference instant wave-free ratio (R-iFR) data set.

The ANN may be trained in two steps involving a first training step in which the R-MPI data set is combined with the R-ICA data set such that an intermediately trained ANN is formed, and a second training step in which the R-iFR data set and/or R-FFR data set is combined with the intermediately trained ANN such that the ANN is generated.

The patient-specific auxiliary parameter data set may comprise age, sex, angina pectoris, and dyspnea. In addition or instead, the patient-specific auxiliary parameter data set may comprise smoking habits, systolic blood pressure, total cholesterol and place of residence.

According to a third aspect it is provided a computer program comprising instructions which, when the program is executed by a computer, cause the computer to carry out the method according to the first aspect.

### BRIEF DESCRIPTION OF THE DRAWINGS

Further objects, features and advantages of the present invention will appear from the following detailed description of the invention, wherein examples of the invention will be described in more detail with reference to the accompanying drawings, in which:

FIG. 1A shows data from a 61 y.o. male with typical angina and an ESC pre-test score of 44, wherein the images from stress supine (upper) and stress upright (lower) show an uptake defect in LAD.

FIG. 1B shows confirmation of an obstruction in the ICA with a QCA value of 92%, which the AI algorithm predicts a QCA value of ≥90%.

FIG. 2 shows the neural network architecture used to predict the stenosis for the three vessels. The illustration shows the spatial size and number of channels for each layer, as well as where the auxiliary parameters are inserted. Here CX denotes a convolution with a X×X filter, followed by a ReLU activation function. The symbol M denotes the occurrence of 2×2max pooling layers. After the final C1 layers (i.e. fully connected), a sigmoid activation function is used.

FIG. 3A-C shows ROC curves when predicting whether the stenosis was above ≥50%, ≥70% and ≥90%, using all auxiliary parameters as well as the two MPI images as input to the CNN.

FIG. 4 shows a block diagram of an example of the present invention and it illustrates the workflow with collection of the MPI images from a cardiac CZT camera, collection of patient characteristics, collection of QCA values from ICA, development and training the CNN-model for prediction of QCA, and finally the use of the trained CNN-model in the clinical praxis.

FIG. 5 shows a block diagram of an example of the present invention, illustrating the three main parts: Input data from MPI stress images in the supine and upright positions, as well as additional patient information. Creation of the algorithm using the data. Prediction of the QCA score.

FIG. 6 is a flowchart illustrating a method for estimating narrowings in arteries of a heart based on sample myocardical perfusion imaging (S-MPI) using an artificial neural network (ANN).

FIG. 7 generally illustrates how the ANN is trained and applied.

FIG. 8 generally illustrates a data processing apparatus configured to estimate narrowings in the arteries of the heart based on the S-MPI data set using the ANN.

### DETAILED DESCRIPTION

Various embodiments of the present disclosure relate generally to medical imaging and related methods. More specifically, particular embodiments of the present disclosure relate to a system and a method for risk assessment in patients with suspected coronary artery disease (CAD) from myocardial perfusion imaging (MPI), by means of deep learning algorithms.

Examples of the present invention will be described more fully hereinafter with reference to the accompanying drawings, in which examples of the invention are shown. This invention may, however, be embodied in many different forms and should not be construed as limited to the examples set forth herein. Rather, these examples are provided so that this disclosure will be thorough and complete, and will fully convey the scope of the invention to those skilled in the art. Like reference signs refer to like elements throughout.

As stated above myocardial perfusion imaging (MPI) is one of the most common cardiological examinations performed for diagnosis and risk assessment in patients with suspected coronary artery disease (CAD), providing valuable information on ischemia, myocardial injuries and left ventricular ejection fraction, among others. The technique has seen improvements in recent years with the introduction of cadmium-zinc-telluride (CZT) technology, allowing to perform the scan in shorter times and low dose radiotracer protocols, among other advantages, achieving high diagnostic performance at the same time. Another option is to use positron emission tomography (PET) for generating image data. Still an option is to use a gamma camera, also referred to as scintillation camera, or an Anger camera.

Patients are usually referred to this study by the cardiologist, who commonly performs a clinical evaluation for ischemia, usually based on the guidelines for the diagnosis and management of chronic coronary syndromes from the European Society of Cardiology (ESC). This information is of great value to nuclear medicine physicians reading the MPI images in order to have a clinical scenario of the patient.

Patients who are thought to have CAD in the MPI study will be further examined, and eventually treated, by means of invasive coronary angiography (ICA). In order to avoid observatory dependent errors and achieve reproducibility, the degree of coronary artery stenosis can be evaluated by means of quantitative coronary angiography (QCA).

Since the pioneer work of Fujita et al in the 1990's, artificial neural networks (ANNs) have been used to evaluate MPI images. ANNs and MPI technology have evolved and recent studies on the field show promising results.

Using ANNs our study aims to predict the degree of coronary artery stenosis from stress MPI images in the upright and supine position, in each of the main coronary artery regions: the left anterior artery (LAD), circumflex artery (LCx) and right coronary artery (RCA), using ICA with QCA as reference. At the same time, we would like to evaluate the improvement of the ANNs in estimating the QCA from MPI images when adding clinical information from the patient such as age, gender, body mass index (BMI) and the ESC pre-test probability scale, which will be regarded as “auxiliary parameters”.

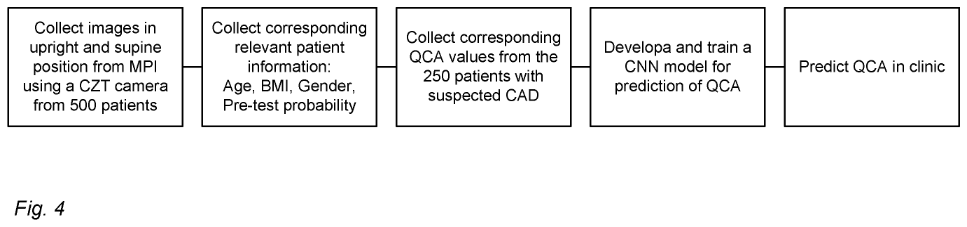

The purpose of the present invention is to evaluate the prediction of quantitative coronary angiography (QCA) values from myocardial perfusion imaging (MPI), by means of deep learning, using invasive coronary angiography with QCA evaluation as the reference method (FIG. 4). It will be shown that deep learning can estimate the QCA percentage of coronary artery stenosis from MPI studies, compared to ICA QCA.

### Materials and Method

To confirm the present invention a study was performed which considered 3058 adult subjects referred to MPI. The referral for stress testing was at the clinical discretion of a cardiologist. Subjects with left-bundle branch block, congenital heart disease and cardiac transplantation were excluded. Due to the risk of bronchospasm from regadenoson all patients with known asthma or chronic obstructive lung disease were asked to perform a lung spirometry test if a PST or a CST was going to be performed, all patients with a forced expiratory volume in 1 second (FEV1)<1 L were excluded from the study. 324 patients underwent ICA within 6 months after the MPI study. From those 324, 263 were evaluated with QCA, including 250 to the study randomly. For the control group we identified and included 250 patients who complied with the above stated inclusion criteria, had no criteria for angina pectoris according to the ESC pre-test probability scale, and were followed up during a period of at least 6 months after the MPI study was performed, showing no cardiovascular events during the follow up period.

### Stress Image Acquisition

MPI was performed according to the European Association of Nuclear Medicine guidelines after a stress test on a CZT camera (“D-SPECT” Spectrum Dynamics, Caesarea, Israel) on the upright and supine positions (1000 stress images) with at least 1 million myocardial counts.

All patients performed either a physical stress test on a bicycle ergometer (BST) (number of patients 265), a pharmacological stress test (PhST) with regadenoson (199), or a combination of both (36), at the discretion of the nuclear medicine physician. All subjects received prior, routine instructions, sent to their home, to avoid potential regadenoson agonists for at least 24 h before MPI (e.g., coffee, tea, cola drinks, chocolate and cacao). In case of use of a PhST, 400 micrograms (5 ml) of regadenoson were administered i.v. In the case of a combined protocol, a BST with 30-50 watts was used with regadenoson after 2 minutes of cycling.

MPI was routinely assessed visually by three different nuclear medicine physicians (each with at least 10 years' experience in MPI) to assess left ventricular myocardial stress perfusion during stress and rest and the degree to which the deficit was reversible, according to current guidelines. All MPI images were retrospectively re-evaluated by an experienced nuclear medicine physician.

### Invasive Coronary Angiography

Invasive coronary angiography was routinely performed according to standard techniques. Percent lumen area reductions due to intracoronary atheromatous plaques were first determined visually on end-diastolic frames and with the help of a quantitative angiography software (QCA) (General Electric Advantage Workstation, Cardiac X-Ray Applications, Stenosis Analysis) for those stenosis visually determined to be around the 50% threshold by an experienced angiographer physician. Where applicable, two separate measurements in orthogonal views of the same stenotic segment were obtained and values were averaged to represent an approximate measurement of the percent (%) vessel area stenosis. Any stenosis ≥50% was considered significant and regarded as a positive QCA test. Total coronary vessel occlusions were marked as “100%” lumen area stenosis. When no visible stenotic lumen was seen on angiography with a marginally patent vessel (With other than normal flow) the stenosis was also regarded as a total occlusion as well.

### Image Processing

Left ventricular (LV) myocardial contours were computed using standard Cedars-Sinai Medical Center Quantitative Perfusion SPECT software. LV contours were defined by a technologist with >15 years of experience in nuclear cardiology who was blinded to angiographic and clinical findings. When needed, the technologist corrected the gross initial LV localization, the LV mask, and the valve plane position.

### Data Analysis

All patients had MPI images from stress in upright and supine position. For some of the patients, artefacts from e.g. breast or diaphragm were visible, but these were included anyway without any special treatment. The following auxiliary parameters were available: BMI, age, gender and ESC pre-test probability for CAD (Table 1).

TABLE 1
Patients characteristics. Values are
n (%) or mean ± standard deviation.

| | No Obstructive | | Obstructive | |
|---|---|---|---|---|
| | Coronary Artery | | Coronary Artery | |
| | Disease | | Disease | |
| Number | 250 | | 250 | |
| 1-vessel disease | — | | 96 | (38%) |
| 2-vessel disease | — | | 62 | (25%) |
| 3-vessel disease | — | | 92 | (37%) |
| Age, yrs. | 63.5 ± 11.6 | | 68.2 ± 9.6 | |
| Male | 144 | (58%) | 187 | (75%) |
| Female | 106 | (42%) | 63 | (25%) |
| BMI | 27.3 ± 4.6 | | 28.4 ± 4.4 | |
| Exercise MPI | 149 | (60%) | 116 | (46%) |
| Pharmacological MPI | 93 | (37%) | 106 | (42%) |
| Combination of exercise and | 8 | (3%) | 28 | (11%) |
| pharmacologic MPI | | | | |
| Pre-test prob. < 15% | 250 | (100%) | 19 | (8%) |

| | | | |
|---|---|---|---|
| 15% ≤ Pre-test prob. ≤ 30% | 0 | 62 | (25%) |
| Pre-test prob. > 30% | 0 | 169 | (68%) |

| | | |
|---|---|---|
| Pre-test prob. | 0 ± 0 | 36.6 ± 14.9 |

In 8 cases the BMI value was missing, in which cases the average BMI-value of the remaining patients was used instead. The data was split into five parts and 5-fold cross-validation was used. The partitioning was done such that there were 50 pathological examples and 50 examples without disease in each fold, but otherwise randomly. The number of examples with stenosis in different intervals can be seen in Table 2.

TABLE 2
Distribution of obstructed coronary arteries
with QCA-values in different intervals.

| QCA interval | LAD | RCA | LCx |
|---|---|---|---|
| 50-59% | 9 | 0 | 1 |
| 60-69% | 17 | 9 | 7 |
| 70-79% | 15 | 6 | 12 |
| 80-89% | 33 | 24 | 28 |
| 90-100% | 54 | 82 | 53 |

### Data Preprocessing

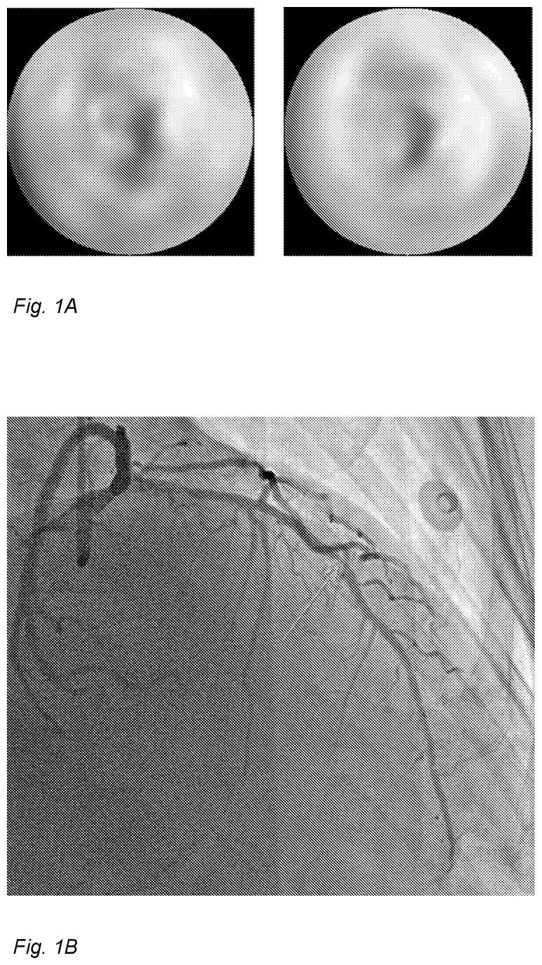

The MPI images were available in the DICOM format. The polar maps were cropped to have the size 296×296 pixels (FIG. 1A). The auxiliary parameters BMI, age and ESC pre-test probability scale where standardized by subtraction of the average and division by the standard deviation (Table 1), in order to improve training of the neural network. When training a deep learning algorithm, augmentation is often used to reduce the risk of overfitting. In previous work, it has been found that augmentation did not improve the results, wherefore this was not used in this particular study. However, if tuned properly, augmentation may be used for improving the results. Even though the auxiliary parameters are exemplified above by BMI, age and ESC pre-test probability scale, other combinations of auxiliary parameters may also be used. For instance, another combination of auxiliary parameters may comprise age, sex, angina pectoris and dyspnea.

### Neural Network

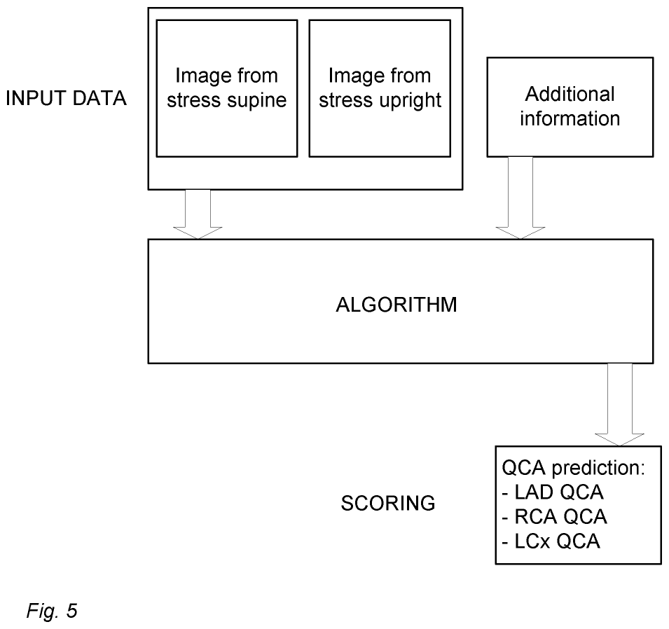

The aim of the algorithm was to estimate the degree of coronary artery stenosis in each of the three main coronary arteries compared to QCA and estimate the improvement of the algorithm when adding information regarding the auxiliary parameters. Therefore, the following settings were used. As input to the neural network the intensity images from stress in upright and supine position were used, stacked as a two-channel image. As additional input, the three auxiliary parameters BMI, age and gender could be included, as well as the pre-test probability according to the ESC scale, to evaluate if these parameters would improve the result. To handle the multiple Independent classes, this was treated as a multi-label problem, where multiple or none of the classes could be the expected output (FIG. 5).

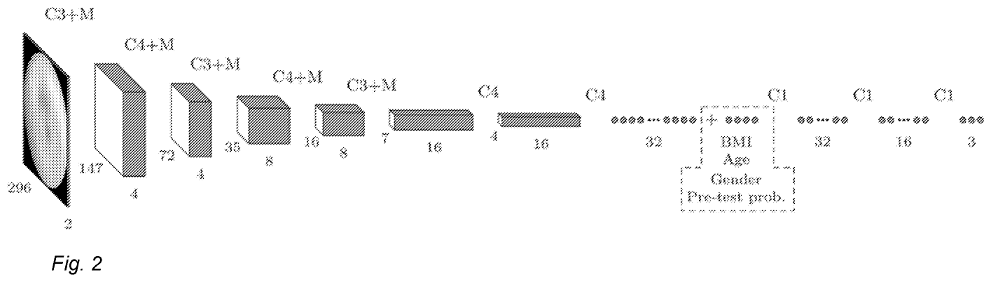

In one example of the present invention, the design of the neural network consists mainly of 3×3 and 4×4 convolutional layers as well as 2×2 max-pooling layers and ReLU activations (FIG. 2). The convolutional part of the neural network, used to extract features from the images, is concatenated with a second input layer where the auxiliary parameters are introduced. Note that the fully connected part of the network has a concatenation layer to include the three or four auxiliary parameters. Dropout was used before the two first C1 layers during training. For the main task, to predict whether the stenosis was above the 50% threshold, one such network was trained. To further predict the percent vessel area stenosis, identical networks were also trained to predict whether the stenosis was above the thresholds 60%, 70%, 80% and 90%. The number of examples in each of these intervals can be seen in Table 2.

The neural network was implemented in Keras and was trained from scratch for 50 epochs with a batch size of 32 using the Adam optimizer with default parameters. Averaged binary cross-entropy was used as loss function to train the model for the multi-label task.

To compensate for the unbalance in class occurrences the loss was weighted in the following way:
Let y_{t,i} denote the true label for region
   i∈{0,1,2} of an example, i.e., whose value is 0 if the region is normal and 1 otherwise. Let y_{p,i} denote the corresponding predicted label, such that 0≤y_{p,i}≤1. Furthermore, let T denote the total number of examples and tᵢ denote the total number of positive examples from region i, that is, the examples with y_{t,i}=1. Define (fixed) weights wᵢ=tᵢ/T, one for each region, and assign to each example and each region a weight ŵᵢ according to the formula

$$\hat{{w}_{i}} = \frac{{y}_{t , i} ( 1 - {w}_{i} ) + ( 1 - {y}_{t , i} ) {w}_{i}}{2 {w}_{i} ( 1 - {w}_{i} )} . \qquad ( ( 1 )$$

Note that the sum of the weights ŵᵢ over all the tᵢ positive training examples is T/2, which equals the sum over all the T−tᵢ negative training examples, as wanted. The total sum over all T training examples is thus T for each of the three regions, giving them equal importance. Finally, the weighted binary loss L_{w} (per example) is defined by

$${L}_{w} = - \frac{1}{3} {\sum }_{i = 0}^{2} \hat{{w}_{i}} [ {y}_{t , i} log {y}_{p , i} + ( 1 - {y}_{t , i} ) log ( 1 - {y}_{p , i} ) ] \qquad ( ( 2 )$$

where the weights ŵᵢ are given by (1) above.

Similar results could be obtained with other designs of the neural network. For example, the convolutional layers could have other sizes, e.g. 2×2, 5×5 or even larger. The number of convolutional layers could also be changed, e.g. adding or removing some fully connected layers in the end of the network. Also, some of the max pooling layers could have larger sizes or be removed. The number of features in each layer could be either increased or decreased, and the performance for these different configurations would depend on e.g. how much data that is used for training. Other types of connections in the network, such as e.g. residual blocks and/or inception blocks could also be used. Regarding training of the network, this could be varied a lot with similar results. Multiple optimizers, including the Adam optimizer which we have used, will give very similar results. Increase or decrease the number of epochs for which the network is trained or the batch size used, would also be possible with similar results. To predict the percentage of stenosis, one could use other approaches than the classifier we have used. One option would be to train the network for regression instead. One could also increase the number of output nodes, to detect multiple intervals with the same network, or use different networks to predict QCA in the three regions (LAD, LCx, RCA).

Even though different designs of the neural network may be applied, it has been found that seven or more layers in the neural network, i.e. the artificial neural network (ANN), can be used to achieve reliable results.

### Results

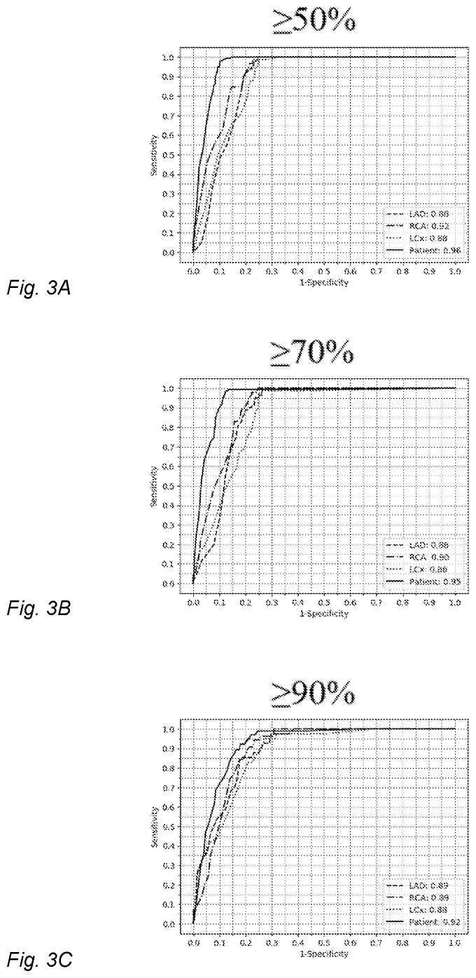

An overview of the area under the receiver-operating characteristic curve (AUC) for the prediction of QCA, with a 50% narrowing of the artery, can be seen in Table 3, both with and without different auxiliary parameters. As it can be seen, the ESC pre-test probability improves the results significantly. Since the result per patient is higher than the average result per vessel, we can draw the conclusion that the majority of the misclassifications occur for the patients with at least one region with QCA≥50%, for which either too many or the wrong regions are predicted to have QCA≥50%. The results for other thresholds of the stenosis can be seen in Table 4. Details of the number of patients in the different intervals can be seen in table 2. The receiver-operating characteristic (ROC) curves for the different regions and the thresholds 50%, 70% and 90% can be seen in FIG. 3.

TABLE 3
AUC results when predicting quantitative coronary angiography
(QCA > 50%) excluding or including the different auxiliary
parameters for the different regions (LAD, RCA, LCx), the average
for the three regions as well as prediction combining all of
them. The standard deviation is given in parenthesis.

| | AUC | | | | |
|---|---|---|---|---|---|
| Settings | LAD | RCA | LCx | Average | Patient |
| No auxiliary | .73 (.04) | .82 (.04) | .79 (.04) | .78 (.02) | .83 (.04) |
| param. | | | | | |
| Incl. age, | .76 (.05) | .85 (.05) | .81 (.04) | .81 (.03) | .86 (.04) |
| sex, gender | | | | | |
| Incl. all | .83 (.03) | .87 (.04) | .86 (.01) | .85 (.02) | .94 (.02) |
| auxiliary | | | | | |
| param. | | | | | |

ANNs have been applied to create an algorithm for the automatic prediction of QCA from stress MPI polar maps and compared it with QCA from ICA, which is the gold standard for diagnosis. While other authors have developed machine learning for the prediction of obstructive coronary artery disease using MPI data, this is the first study with deep learning that works with the prediction of QCA from stress MPI (FIG. 1).

The implications of this findings for the health care system are sustainable since the information provided from the QCA, by means of the MPI, using a non-invasive approach, could result in advantages like avoiding the ICA intervention, which would in turn be translated in less radiation exposure to the patients, less hospitalizations and less stress for the healthcare system as a whole.

According to the literature, the current hierarchical approach of the definition with respect to clinically indicated repeat revascularization is as follows: 1; QCA (preferably three-dimensional (3D) QCA) diameter stenosis ≥50% (based on the average of multiple views) with either recurrent symptoms or positive non-invasive functional test. 2; QCA (preferably 3D QCA) diameter stenosis >70% (based on the average of multiple views) regardless of other criteria. 3; QCA diameter stenosis >70% (based on the worst view) regardless of other criteria. The developed algorithm can predict coronary artery stenosis of 50, 60 and 70% with high accuracy (see Table 4).

TABLE 4
AUC results when predicting QCA-value for the different coronary artery
regions (LAD, RCA, LCx), the average for the three regions as well
as prediction combining all of them. The standard deviation is given
in parenthesis, and all auxiliary parameters were included.

| | AUC | | | | |
|---|---|---|---|---|---|
| QCA-limit | LAD | RCA | LCx | Average | Patient |
| 50% | .83 (.03) | .87 (.04) | .86 (.01) | .85 (.02) | .94 (.02) |
| 60% | .82 (.02) | .88 (.03) | .85 (.02) | .85 (.02) | .94 (.01) |
| 70% | .82 (.03) | .87 (.03) | .85 (.04) | .85 (.02) | .93 (.02) |
| 80% | .83 (.02) | .86 (.04) | .85 (.04) | .85 (.02) | .92 (.02) |
| 90% | .81 (.05) | .85 (.04) | .80 (.03) | .82 (.01) | .88 (.02) |

Thus, it could play an important role in the clinical scenario in the future if fully developed.

We also observed that the addition of information from the “auxiliary parameters” to the deep learning algorithm improves CAD prediction, on both per-patient and per-vessel basis (see Table 3). While the improvement when adding age, sex and gender was non-significant, the pre-test probability according to the ESC scale gave a significant improvement. This highlights the importance of a good clinical history from the cardiologist or the managing doctor and that this has to be communicated to the nuclear medicine specialist, as this is precious information when reading the MPI images.

We demonstrated that deep learning achieves high diagnostic efficacy in predicting the percentage of coronary artery stenosis compared to QCA. The majority of the errors by the deep learning algorithm occur for patients with at least one narrowed vessel, where the wrong vessel or too many vessels are predicted positive by the algorithm.

Thus, in the present invention, using ANNs, the algorithm can estimate the percentage of coronary artery stenosis from MPI images, compared to ICA QCA, achieving very satisfactory results. The algorithm improves its performance when adding information regarding pre-test probability for CAD from the ESC guidelines.

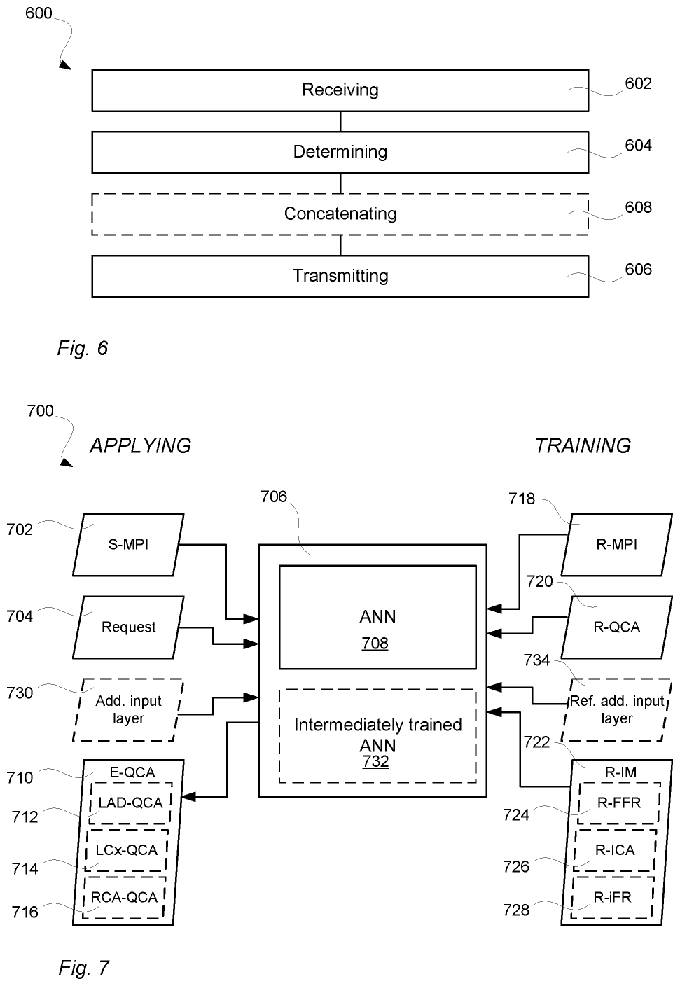

FIG. 6 is a flowchart illustrating a method 600 for estimating narrowings in arteries of a heart based on a sample myocardial perfusion imaging (S-MPI) data set 702 using an artificial neural network (ANN) 708. In a first step 602, the S-MPI data set 702 and a request 704 are received. In a second step 604, an estimate quantitative coronary angiography (E-QCA) data set 710 is determined based on the S-MPI data set 702 using the ANN 708. As illustrated in FIG. 7, the ANN 708 can be trained by using a reference MPI (R-MPI) data set 718, a reference quantitative coronary angiography (R-QCA) data set 720 and a reference invasive measurement (R-IM) data set 722. Each record in the R-MPI data set 718 can have a corresponding record in the R-QCA data set 720 and a corresponding record in the R-IM data set 722, respectively. In a third step 606, the E-QCA data set 710 is transmitted in response to the request 704.

Optionally, in a fourth step 608, a convolutional part of the ANN 708 can be concatenated with an additional input layer 730, wherein the convolutional part is used for extracting features from the S-MPI data set 702. The additional input layer 730 may comprise a patient-specific auxiliary parameter data set.

FIG. 7 illustrates an overview 700 of data sets used for training the ANN 708, which may be held in a data processing apparatus 706, as well as data sets used when applying the ANN 708.

When applying the ANN 708, the S-MPI data set 702 can be fed to the ANN 708 together with a request 704. The request 704 may comprise, in addition to information required from a data communications perspective, information of the party requesting the analysis of the S-MPI data set 702, an identifier linked to a patient to make sure that the S-MPI data set 702 can later on be linked to e.g. the patient-specific auxiliary parameter data set etc. Even though illustrated and discussed as two separate data sets, these two may also be made into one and the same data set.

After having processed the S-MPI data set 702 as described above, the estimate quantitative coronary angiography (E-QCA) data set 710 can be output and provided to a physician or other medical staff or another piece of software for further analysis. The E-QCA data set 710 may comprise a left anterior artery (LAD) QCA data sub-set 712, a circumflex artery (LCx) QCA data sub-set 714 and a right coronary artery (RCA) QCA data sub-set 716.

When training the ANN 708, a reference MPI (R-MPI) data set 718, a reference quantitative coronary angiography (R-QCA) data set 720 and a reference invasive measurement (R-IM) data set 722 may be taken into account. Each record in the R-MPI data set 718 can have a corresponding record in the R-QCA data set 720 and a corresponding record in the R-IM data set 722, respectively.

The R-IM data set 722 may comprise a reference fractional flow reserve (R-FFR) data set 724, an reference invasive coronary angiography (R-ICA) data set 726 and/or an reference instant wave-free ratio (R-iFR) data set 728.

An additional input layer 730 may also be provided when applying the ANN 708. The patient-specific auxiliary parameter data set may be comprised in this additional input layer 730. As described above, this additional input layer 730 may be used for improving the extraction of features from the S-MPI data set 702. A reference additional input layer 734 may be used during training.

As described above, the ANN may be trained in two steps involving a first training step in which the R-MPI data set 718 is combined with the R-ICA data set 726 such that an intermediately trained ANN 732 is generated, and a second training step in which the R-iFR data set 728 and/or the R-FFR data set 724 is combined with the intermediately trained ANN 732 such that the ANN 708 is generated

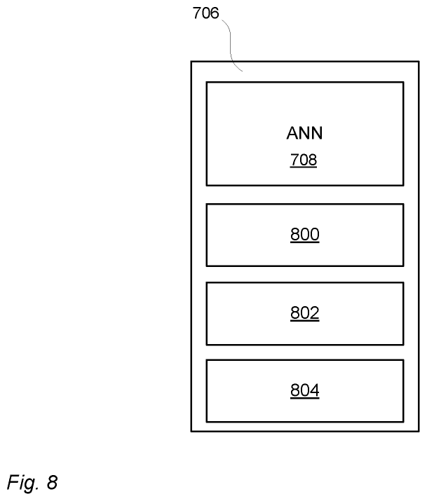

FIG. 8 schematically illustrates the data processing apparatus 706 discussed above. In addition to the ANN 708, and possibly also, even though not illustrated, the intermediately trained ANN 732, the apparatus 706 may comprise a data receiver 800 configured to receive the S-MPI data set 702 and the request 704. In addition, a processor 802 configured to determine the E-QCA) data set 710 based on the S-MPI data set 702 using the ANN 708 can be provided. A data transmitter 804 configured to transmit the E-QCA data set 710 in response to the request 704 can also be provided.

The terminology used herein is for the purpose of describing particular examples only and is not intended to be limiting of the invention. As used herein, the singular forms “a”, “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms used herein should be interpreted as having a meaning that is consistent with their meaning in the context of this specification and the relevant art and will not be interpreted in an idealized or overly formal sense unless expressly so defined herein.

The foregoing has described the principles, examples and modes of operation of the present invention. However, the invention should be regarded as illustrative rather than restrictive, and not as being limited to the particular examples discussed above. The different features of the various examples of the invention can be combined in other combinations than those explicitly described. It should therefore be appreciated that variations may be made in those examples by those skilled in the art without departing from the scope of the present invention as defined by the following claims.

1. A computer-implemented method for estimating narrowings in arteries of a heart based on a sample myocardial perfusion imaging (S-MPI) data set using an artificial neural network (ANN), said method comprising:
receiving, in a data processing apparatus, the S-MPI data and a request,
determining, in the data processing apparatus, an estimate quantitative coronary angiography (E-QCA) data set based on the S-MPI data set using the ANN, wherein the ANN is trained using a reference MPI (R-MPI) data set, a reference quantitative coronary angiography (R-QCA) data set and a reference invasive coronary angiography (R-ICA) data set, wherein each record in the R-MPI data set has a corresponding record in the R-QCA data set and a corresponding record in the R-ICA data set, respectively, and
transmitting, from the data processing apparatus, the E-QCA data set in response to the request.

2. (canceled)

3. (canceled)

4. The computer-implemented method according to claim 1, wherein the ANN is a convolutional neural network (CNN).

5. The computer-implemented method according to claim 1, wherein a patient-specific auxiliary parameter data set is comprised in an additional input layer, said method further comprising:
concatenating a convolutional part of the ANN with the additional input layer, wherein the convolutional part is used for extracting features from the S-MPI data set.

6. The computer-implemented method according to claim 5, wherein the patient-specific auxiliary parameter data set comprises age, sex, angina pectoris, and dyspnea.

7. The computer-implemented method according to claim 1, wherein the ANN comprises at least 7 layers.

8. The computer-implemented method according to claim 1, wherein the S-MPI data set is captured by using a cadmium zinc telluride (CZT) camera, a gamma camera, and/or a positron emission tomography (PET) camera.

9. The computer-implemented method according to claim 1, wherein the E-QCA data set comprises a left anterior artery (LAD) QCA data sub-set, a circumflex artery (LCx) QCA data sub-set and a right coronary artery (RCA) QCA data sub-set.

10. The computer-implemented method according to claim 1, wherein the S-MPI data set and the R-MPI data set comprise stress MPI data.

11. A data processing apparatus for estimating narrowings in arteries of a heart based on a sample myocardial perfusion imaging (S-MPI) data set using an artificial neural network (ANN), said apparatus comprising:
a data receiver configured to receive the S-MPI data set and a request,
a processor configured to determine an estimate quantitative coronary angiography (E-QCA) data set based on the S-MPI data set using the ANN, wherein the ANN is trained using a reference MPI (R-MPI) data set, a reference quantitative coronary angiography (R-QCA) data set and a reference invasive coronary angiography (R-ICA) data set, wherein each record in the R-MPI data set has a corresponding record in the R-QCA data set and a corresponding record in the R-ICA data set, respectively, and
a data transmitter configured to transmit the E-QCA data set in response to the request.

12. (canceled)

13. (canceled)

14. The data processing apparatus according to claim 11, wherein a patient-specific auxiliary parameter data set comprises age, sex, angina pectoris, and dyspnea.

15. A computer program comprising instructions which, when the program is executed by a computer, cause the computer to carry out the computer-implemented method of claim 1.

16. The data processing apparatus according to claim 11, wherein a patient-specific auxiliary parameter data set is comprised in an additional input layer, and said processor is further configured to concatenate a convolutional part of said ANN with the additional input layer, wherein the convolutional part is used for extracting features from the S-MPI data set.
